(19) United States
(12) Patent Application Publication
(10) Pub. No.: US 2010/0216719 A1
(43) Pub. Date: Aug. 26, 2010

(54) Soluble Fiber Combinations for Weight Control and Improving Parameters of Cardiovascular Health

(75) Inventor: Robert A. Sinnott, Keller, TX (US)

Correspondence Address: CHALKER FLORES, LLP, 2711 LBJ FRWY, Suite 1036, DALLAS, TX, 75234, US

(73) Assignee: MANNATECH, INCORPORATED, Coppell, TX (US)

(21) Appl. No.: 12/758,611

(22) Filed: Apr. 12, 2010

Related U.S. Application Data
(62) Division of application No. 11/539,796, filed on Oct. 9, 2006.
(60) Provisional application No. 60/725,269, filed on Oct. 11, 2005.

(51) Int. Cl. A61K 38/16; A61K 31/715

(52) U.S. Cl. 514/12; 514/54

(57) ABSTRACT

A food or pharmaceutical agent is provided for treating or offsetting the risks of a disease or disorder such as a cardiovascular disease, a hypercholesterolemia disorder, a low serum high density lipid (HDL)/low density lipid (LDL) ratio, a hypertriglyceridemia disorder, and diabetes includes a pharmaceutical carrier, and a composition including a purified glucomannan and at least one galactomannan. A method for treating or offsetting the risks of such a disease or disorder includes the step of administering to a human in need thereof an effective amount of the pharmaceutical composition to treat the cardiovascular disease.

### CROSS-REFERENCES TO RELATED APPLICATIONS

This application claims the benefit of U.S. Provisional Application No. 60/725,269, filed Oct. 11, 2005, which is incorporated herein by reference.

### FIELD OF THE INVENTION

The present invention generally relates to dietary and therapeutic formulations that include natural, substantially indigestible, long-chain carbohydrates. More particularly, the present invention relates to the use of such formulations in dietary and therapeutic regimens.

### BACKGROUND OF THE INVENTION

Hyperlipidemia is a known risk factor for the development of atherosclerosis, coronary artery disease, and myocardial infarction. Hypercholesterolemia is a pathogenic factor for arteriosclerosis, stroke, heart disease, and angina pectoris. Consequently, reducing plasma lipid concentrations, serum cholesterol concentrations, and serum triglyceride concentrations is a priority for combating the onset and progression of cardiovascular disease. Plasma lipid reduction is routinely accomplished by employing dietary modifications and/or by intake of pharmaceutical drugs such as the statin family of drugs, which inhibit the synthesis of cholesterol in the liver.

Glucomannan is an indigestible carbohydrate, which in contact with water, can hydrate to form a viscous gel (i.e. hydrated polymer complex). This viscous gel can inhibit the enzymatic digestion of food by reducing contact between digestive enzymes and food substances and also diminish subsequent absorption of digested food in the G.I tract. High chain length glucomannan polymers exist. These high chain length polymers hydrate to form extremely viscous gels within minutes after mixing with sufficient water. Since inhibition of enzymatic digestion and reduced food absorption is based on the rheological properties of the gel, the more viscous gels are highly desirable for weight control formulations. Therefore, high molecular weight polymers are desirable as the basis of soluble fiber formulations for the purpose of weight control.

When a formulation containing a high molecular weight glucomannan polymer is consumed prior to eating a meal, it temporarily suppresses digestion and absorption of calories, carbohydrates, protein and fat from food. The formulation also enhances excretion and elimination of cholesterol derivitives (i.e. neutral sterols and bile) from the body. Thus, similar beneficial effects of portion control or reduced caloric intake are provided by formulations containing a high molecular weight glucomannan polymer. Furthermore, rather than having to restrict caloric intake by consuming smaller amounts of food, a person may consume usual amounts of food with less food being digested and absorbed into the body. It is proven that reduction of caloric intake via portion control is proven to be the best way to accomplish and maintain long-term weight loss. By using glucomannan polymers effectively in weight control formulations, a similar effect to portion control may be achieved without participating in conventional dieting.

In addition to the weight loss-related advantages provided by glucomannan polymers, it has been demonstrated in humans that ingestion of soluble fibers such as glucomannan polymers, under certain conditions, may positively affect measurable parameters of cardiovascular health. Such effects may include reduced levels of serum cholesterol, triglycerides, and glucose. Chen et. al., J. of the American College of Nutrition, 22:36-42 (2003) demonstrates that subjects supplemented with 3.8 grams per day of Konjac glucomannan for 28 days experienced an 11.1% reduction in plasma cholesterol, a 20.7% reduction in LDL cholesterol, a 15.6% reduction in total cholesterol/HDL cholesterol ratio, a 12.9% reduction in ApoB, and a 23.2% reduction in fasting glucose levels. Other controlled metabolic trials, e.g. Vuksan et. al., Diabetes Care, 22:913-919 (1999) and Vuksan et. al., Diabetes Care, 23:9-14 (2000), demonstrate that diets rich in Konjac glucomannan may improve glycemic control, blood pressure, and lipid profile in humans.

Glucomannans are not the only gel-forming, indigestable carbohydrate polymers in the human diet. Other classes of carbohydrate-type polymers (i.e. soluble fibers) that are frequently found in human diets include cellulose gums, pectins, galactomannans, beta-glucans and others. While some of these other soluble fibers may form viscous hydrated gels like glucomannan, they do not necessarily function in the same way. For example, various types of soluble fibers may differ greatly in their ability to affect measurable parameters of cardiovascular health. This may be due in part to the different chemical structures of the soluble fibers and the different rheological properties of the soluble fibers, particularly, their ability to form viscous gels upon hydration. Talukdar et. al., J. of Pharmaceutical Science, 85:537-540 (1996) details the various rheological properties of a natural soluble fiber, xanthan gum, and a synthetic soluble fiber, hydroxypropylmethyl cellulose, as they relate to controlled release of drug compounds. In this study xanthan gum was found to have superior drug release characteristics under certain conditions because of their markedly different rheological properties. Even different preparations of the same type of soluble fiber may have markedly different rheological properties. For example, depending on how a source plant is cultivated and processed, konjac glucomannan preparations may range from low viscosity (short molecular weight polymers) to very high viscosity (long molecular weight polymers).

According to a study disclosed in Soh, et. al. J. of Medicinal Foods, 6:225-230 (2003), soluble fibers from food and bacterial sources had measurably different cholesterol adsorption capacities this suggests that soluble fiber compounds are not all equal for therapeutic development purposes. In another study disclosed in Levrat-Verny et. al, J. of Nutrition, 130:600-607 (2000), xanthan gum, one type of galactomannan soluble fiber, significantly reduced both cholesterol and triglycerides in test animals while guar gum, another type of galactomannan soluble fiber, decreased only cholesterol in test animals. In one clinical study, a combination of galactomannans (i.e. guar gum and xanthan gum in 1:3 ratio) performed better than either product alone or other combinations of galactomannans for improving certain health parameters such as, serum triglycerides, cholesterol, HDL/LDL ratio, serum glucose levels, etc. In another study reported in Gallaher et. al., J. of Nutrition, 130:2753-2759 (2002), a combination of chitosan and glucomannan in a 1:1 had a hypocholesterolemic effect in human subjects consuming 2.4 grams per day of this combination for 28 days.

With all the potential beneficial health effects provided by soluble fibers, there is a need for a well designed, optimized product containing a combination of dietary fiber ingredients, alone and in combination with insoluble fibers and other compositions. There is also a need for such compositions produced in a manner that would represent a cost saving to consumers. Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A food or pharmaceutical agent is provided for treating or offsetting the risks of a disease or disorder such as a cardiovascular disease, a hypercholesterolemia disorder, a low serum high density lipid (HDL)/low density lipid (LDL) ratio, a hypertriglyceridemia disorder, and diabetes. The food or pharmaceutical agent includes a composition including a purified glucomannan and at least one galactomannan. A pharmaceutical carrier is also included if the composition is intended for pharmaceutical purposes. The food or pharmaceutical agent may also include additional soluble and/or insoluble fibers.

A method is also provided for treating of offsetting the risks of a disease or disorder such as a cardiovascular disease, a hypercholesterolemia disorder, a low serum high density lipid (HDL)/low density lipid (LDL) ratio, a hypertriglyceridemia disorder, and diabetes. The method includes the step of administering to a human in need thereof an effective amount of a food or pharmaceutical composition to treat or offset the risks of the cardiovascular disease, the composition including a purified glucomannan and at least one galactomannan. The pharmaceutical agent may also include additional soluble and/or insoluble fibers.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The present invention relates generally to formulations that include natural, indigestible, long-chain carbohydrates including soluble fiber. Some of such formulations are capable of facilitating weight loss and/or improving cardiovascular health by lowering serum cholesterol concentrations, lowering serum triglyceride concentrations, raising HDL/LDL cholesterol ratios, reducing blood glucose levels after meals, stabilizing blood glucose levels between meals, lowering inflammatory mediator concentrations, and lowering blood pressure in a human. Such formulations also have positive effects on measurable parameters of oxidative stress, inflammation and aging.

Cardiovascular disorders and other health conditions such as those listed above may be signs of disease processes and are conventionally treated by administering suitable drugs. Soluble fiber formulations of the present invention are used to supplant the need for such drugs as either a dietary supplement or a food additive. According to specific embodiments of the invention, the soluble fiber compositions are used to reduce the daily requirement of such drugs to adequately control disease conditions such as hypercholesterolemia, hyperlipidemia, hypertension, diabetes, prediabetic syndrome in adults and children, and diabetic predisposition in adults and children.

The pharmaceutical agents of the present invention include carriers such as pills, capsules, tablets, powders, and liquids. Combined with the pharmaceutical carriers are compositions that include purified glucomannan and purified galactomannan. Glucomannan is a water-soluble dietary fiber, and constitutes about 40% by dry weight of the roots or corm of the konjac plant. The polysaccharide consists of glucose and mannose in a proportion of 5:8 joined by (1-4) beta linkages. The basic polymeric repeating unit of glucomannan has the pattern: GGMMGMMMMMGGM. Short side chains of eleven to sixteen monosaccharides occur at intervals of 50 to 60 units of the main chain, and are attached thereto by (1-3) beta linkages. Also, acetate groups are located every nine to nineteen units of the main chain. Galactomannan compounds are polysaccharides having a mannose backbone with galactose side groups. More specifically, the galactomannan structure includes a (1-4)-linked beta-D-mannopyranose backbone with branchpoints from their 6-positions linked to alpha-D-galactose, i.e. 1-6-linked alpha-D-galactopyranose. Some exemplary galactomannan compounds include fenugreek gum, which has a mannose:galactose ratio of about 1:1, guar gum, which has a mannose:galactose ratio of about 2:1, tara gum, which has a mannose:galactose ratio of about 3:1, and locust bean gum or carob gum, which has a mannose:galactose ratio of about 4:1.

According to one exemplary embodiment, the food additive or pharmaceutical composition includes the purified konjac glucomannin together with one galactomannan component. However, in a preferred embodiment a plurality of galactomannan components are included with the konjac glucomannin. This is because recent discoveries demonstrate that ingestion of certain dietary fiber combinations appear to be preferable to ingestion of single types of soluble fiber for modifying certain health parameters in humans. Because of different and perhaps complementary mechanisms of action, combinations of different glactomannans together with the glucomannan provide additive, and sometimes synergistic, effects on the human body.

An exemplary composition includes the galactomannan and glucomannan components as the only as the only fiber constituents. However, other exemplary compositions include one or more additional soluble fibers and/or insoluble dietary fibers. Psyllium gum, xanthan gum, locust bean gum, pectin, inulin, a fungal polysaccharide, oat bran, oat beta-glucan, extracts of beans, extracts of pods, and extracts of foliage of a Leguminoseae family member are just a few examples of additional soluble fibers. Cellulose-based compounds, fungal-derived fiber, mushroom fiber, beet fiber, and chitin-based compounds and derivatives thereof are just a few examples of some insoluble fibers that may be included. The soluble and insoluble fibers are included at ratios that optimize the targeted health effects when consumed at low dosage rates.

According to one embodiment, the composition includes at least one protein. Some exemplary proteins have a low methionine/glycine ratio ranging between about 0.1 and about 0.5. Other exemplary proteins have a low lysine/arginine ratio ranging between about 0.6 and 1.0. According to another embodiment, the composition includes a mixture of at least one plant sterol and at least one plant stanol. Exemplary proteins would include fish protein and soy protein. Exemplary plant sterols and stanols would include sitosterol, campesterol, stigmasterol, and sitostanol.

As previously discussed, the composition may be a food additive or a pharmaceutical agent that is combined, dissolved, or contained in various pharmaceutical carriers. Some carriers are more suitable than others, depending on the required composition dosage and the treatment provided thereby. For many treatments, beneficial effects are provided by two to four capsules containing 200 to 1000 milligrams of soluble fiber components taken orally as part of a daily supplementation regimen. For other treatments, a drink product containing the composition dissolved in water or a water-containing solvent provides a beneficial effect as part of a daily supplementation regimen. Exemplary drink products include the soluble fiber inulin or cellulose gums in addition to the galactomannan and glucomannan fibers and any other soluble or insoluble fiber constituents.

As previously discussed, some soluble fibers provide a cholesterol lowering effect when administered. The glucomannan and galactomannan combinations of the present invention are used as an adjuvant for cardiovascular disease therapy, including hypercholesterolemia therapy, a hypertriglyceridemia therapy, or a therapy for treating a low serum high density lipid (HDL)/low density lipid (LDL) ratio. For example, to decrease the daily maintenance dosage of drugs required to maintain healthy serum cholesterol levels, healthy triglyceride levels, healthy serum HDL/LDL, ratios or to otherwise maintain a healthy cardiovascular system, a human in need is administered an effective amount of a pharmaceutical composition including the purified glucomannan and at least one galactomannan. A preferred composition for such treatments includes a plurality of different galactomannan constituents. An exemplary prescribed therapy includes the composition of the present invention together with a drug, such as a hypercholesterolemia drug, or a hypertriglyceridemia drug.

The inventive formulations also provide diabetes treatments, or treatments for preventing diabetes. The glucomannan and galactomannan combinations are capable of lowering and/or maintaining serum glucose levels. More particularly, the present glucomannan and galactomannan compositions have an ability to lower serum glucose levels by slowing digestion and uptake of carbohydrates and sugars from foods, and consequently have utility as an adjuvant to potentiate or supplant some use diabetic drugs such as insulin. Thus, the daily maintenance dosage of supplemental insulin required to maintain healthy blood glucose levels may be reduced when taken along with a daily regimen of the present glucomannan and galactomannan compositions. As with previously discussed treatments, the present compositions may also include additional soluble and/or insoluble fibers. An exemplary prescribed therapy includes the composition of the present invention together with a diabetes drug such as insulin.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

1-10. (canceled)

11. A pharmaceutical agent or food, comprising:
a carrier; and
a composition in the carrier, comprising a purified glucomannan and at least one galactomannan.

12. The pharmaceutical agent or food according to claim 11, wherein the carrier is selected from the group consisting of a tablet, a capsule, a powder, and a solvent.

13. The pharmaceutical agent or food according to claim 12, wherein the carrier is a solvent, and the composition further comprises inulin.

14. The pharmaceutical agent or food according to claim 11, wherein the only fibers included in the composition consist of the purified glucomannan and the at least one galactomannan.

15. The pharmaceutical agent or food according to claim 11, further comprising a soluble fiber selected from the group consisting of psyllium gum, locust bean gum, pectin, inulin, a fungal polysaccharide, oat bran, oat beta-glucan, an extract of beans, an extract of pods, and an extract of foliage of a Leguminoseae family member.

16. The pharmaceutical agent or food according to claim 11, further comprising an insoluble fiber selected from the group consisting of cellulose-based compounds, fungal-derived fiber, mushroom fiber, beet fiber, and chitin-based compounds and derivatives thereof.

17. The pharmaceutical agent or food according to claim 11, further comprising a protein selected from the group consisting of proteins having a low methionine/glycine ratio of around 0.1 to 0.5 and proteins having a low lysine/arginine ratio of around 0.6 to 1.0.

18. The pharmaceutical agent or food according to claim 11, further comprising a mixture of at least one plant sterol and at least one plant stanol.

19. The pharmaceutical agent or food according to claim 11, further comprising a hypertriglyceridemic drug.

20. The pharmaceutical agent or food according to claim 11, further comprising a hypercholesterolemic drug.

21. The pharmaceutical agent or food according to claim 11, further comprising a diabetes drug.
